(19) United States
(12) Patent Application Publication
(10) Pub. No.: US 2016/0047827 A1
(43) Pub. Date: Feb. 18, 2016

(54) Apparatus for Analyzing the Process of Formation of Aggregates in a Biological Fluid and Corresponding Method of Analysis

(71) Applicants: UNIVERSITA' DEGLI STUDI DI UDINE, Udine (IT); CENTRO DI RIFERIMENTO ONCOLOGICO, Aviano (IT)

(72) Inventors: Francesco Trevisan, Udine (IT); Antonio Affanni, Casalmaggiore (IT); Ruben Specogna, Pagnacco (IT); Luigino De Marco, Maniago (IT); Mario Mazzucato, Concordia Sagittaria (IT); Monica Battiston, Villotta Di Chions (IT)

(73) Assignees: UNIVERSITA' DEGLI STUDI DI UDINE, Udine (IT); CENTRO DI RIFERIMENTO ONCOLOGICO, Aviano (IT)

(21) Appl. No.: 14/782,302

(22) PCT Filed: Apr. 3, 2014
(86) PCT No.: PCT/IB2014/060395

§ 371 Date: Oct. 2, 2015

(30) Foreign Application Priority Data
Apr. 3, 2013 (IT) UD2013A000047

(51) Int. Cl. G01N 33/86; G01N 27/12

(52) U.S. Cl. CPC G01N 33/86; G01N 27/128; G01N 2800/226; G01N 2800/224

(57) ABSTRACT

Apparatus for analyzing the process of formation of aggregates in a biological fluid, such as blood or hematic fluids, comprising a perfusion chamber provided with at least one micro-channel through which the biological fluid flows, and in which at least one reactive substrate is present, such as a cyto-adhesive substrate, to stimulate the aggregation process in the biological fluid, and impedenziometric detection means associated with the micro-channel, in correspondence to at least one investigation area and disposed, during use, in contact with the flow of biological fluid in transit, in order to detect impedance data of the biological fluid. The impedenziometric detection means comprise at least two first electrodes with an oblong development disposed in the micro-channel, and a plurality of second electrodes with an oblong development disposed in the space comprised between the first electrodes and according to a pattern defining substantially the perimeter of a geometric figure.

### FIELD OF THE INVENTION

The present invention concerns an apparatus for analyzing the process of formation of aggregates, such as platelet aggregates, or red blood cells or other in a biological fluid, such as blood or other hematic fluids, either animal or human, and mixtures of said fluid with additive substances. More generally the present invention concerns an apparatus to detect isolating defects in a conductor fluid.

In particular, the present invention provides the “in vitro” study of the growth phenomenon over time of the aggregates in controlled flow conditions of the fluid, in order to assess, for example, the onset of cardiovascular pathologies, as potential thrombotic risks in “silent” subjects, and potential hemorrhagic risks in subjects with congenital and acquired disorders of the hemostasis. Moreover, the apparatus according to the present invention allows to monitor the effectiveness of anti-thrombotic drugs or other drugs.

The present invention also concerns a method to analyze the aggregation process in the biological fluid.

### BACKGROUND OF THE INVENTION

The coagulation process is the physiological mechanism that causes the loss of blood from damaged blood vessels to stop, and it is essential to guarantee the integrity of an individual in the event of a hemorrhage.

Thrombosis is an unwanted formation of a hemostatic plug or thrombus inside a blood vessel or the cardiovascular system.

As is known, the adhesive and aggregative capacity of the platelets and the formation of fibrin normally control the repair of tissue damage. The activation of the platelets causes the formation of a mass of aggregated platelets that form a plug to stop hemorrhagic events.

Sometimes the platelets can exasperate the repair process, as they are activated inappropriately if there is a pathological change in the hemostatic process, arteriosclerosis for example that can lead to a dramatic event which causes the occlusion of the blood vessel: thrombosis.

A pathological hemostatic event, whether it is thrombotic ischemic due to the sudden lack of blood flow in a blood vessel, as in the case of the coronaries in myocardial infarction, or a hemorrhagic event, is a severe problem for the cardiovascular system. These problems derive from cardiovascular alterations acquired over time, such as arteriosclerotic manifestations, aneurisms, arteriovenous shunts, vasculitis, anomalies of the cardiac valves, atrial fibrillation and cardiac conduction diseases, venous and arterial thromboses or other.

Moreover, these pathological manifestations are difficult to diagnose since they are also present in subjects who during their normal life have never had pathological events and who, for example, when undergoing surgical operations, can incur serious hemorrhagic or thrombotic-ischemic manifestations.

To this end, it is fundamentally important, in a clinical environment, to have an “ex-vivo” functional test that allows to identify potential thrombotic and hemorrhagic risks in “silent” subjects for cardiovascular pathological manifestations.

To this end the European patent application 06819957.9 is known, which concerns an apparatus for the diagnosis, prognosis and pharmacological monitoring of the thrombotic-ischemic and hemorrhagic pathology of the cardiovascular apparatus.

In particular, this known apparatus comprises a chamber, also called perfusion chamber, for the passage or flow of hematic liquids.

The perfusion chamber is provided with micro-channels in which, by means of pumping and suction means, the blood which has been taken from the patient into a test tube is made to flow, after having been suitably treated with anticoagulants, such as for example heparin, citrate or similar, and with fluorescent probes that act as optical markers, such as for example quinacrine, phycoerythrin or similar.

Optical acquisition means coordinated with said markers for fluorescence analysis acquire images relating to the progress of the hemostatic processes that lead to the formation of thrombi inside the micro-channels.

A processing unit processes the images acquired and supplies indications relating to the behavior of the patient's hemostasis.

The optical acquisition means can be for example confocal optical microscopes or inverted optical microscopes.

Using an inverted microscope the formation of the thrombus is quantified through a two-dimensional digital image. With this approach, the three-dimensional reconstruction of the formation of the thrombus can only be extrapolated indirectly from the two-dimensional one and might not reflect its real size.

This problem can be solved with a confocal microscope, and on this point see the publications of M. Mazzucato, M. R. Cozzi, P. Pradella, D. Perissinotto, A. Malmstrom, M. Morgelin, P. Spessotto, A. Colombatti, L. De Marco, R. Perris, Vascular, “PG-M/versican variants promote platelet adhesion at low shear rates and cooperate with collagens to induce aggregation” FASEB J. 16 (2002), 1903-1916; by M. Mazzucato, P. Pradella, M.R. Cozzi, L. De Marco, Z. M. Ruggeri, “Sequential cytoplasmic calcium signals in a 2-stage platelet activation process induced by the glycoprotein Ibalpha mechanoreceptor”, Blood 100 (2002) 2793-2800; by M. Mazzucato, M. R. Cozzi, P. Pradella, Z. M. Ruggeri, L. De Marco, “Distinct roles of ADP receptors in von Willebrand factor mediated platelet signaling and activation under high flow”, Blood 104 (2004), 3221-3227; by A. Casonato, L. De Marco, L. Gallinaro, M. Sztukowska, M. Mazzuccato, M. Battiston, A. Pagnan, Z.M. Ruggeri, “Altered von Willebrand factor subunit proteolysis and multimer processing associated with the Cys2362Phe mutation in the B2 domain”, Thrombosis and Hemostasis 97 (2007) 527-533; by M. Mazzucato, M. R. Cozzi, M. Battiston, M. Jandrot-Perrus, M. Mongiat, P. Marchese, T.J. Kunicki, Z.M. Ruggeri, L. De Marco, “Distinct spatio-temporal Ca2+signaling elicited by integrin alpha2f31 and glycoprotein VI under flow”, Blood 114 (2009), 2793-280.

However, the sizes of a confocal microscope, and above all its cost, limit its use to sophisticated research laboratories.

Moreover, since it is a reconstruction in real time, the monochromatic laser source used to illuminate the platelet aggregates during the hemostasis process interacts heavily on the process itself, transferring energy that alters or compromises the dynamics. Simpler and more economic devices are also widely used, but they are less accurate for monitoring cells in flow conditions which integrate fluidic channels located directly on CMOS lensless chips, also functioning in conditions of fluorescence with visual fields higher than cm² and resolution of some microns; see for example U. Gurkan, S. Moon, H. Gecki, F. Xu, S. Wang, T. J. Lu and U. Demirci, “Miniaturized lensless imaging systems for cell and microorganism visualization in point-of-care testing”, Biotechnol. J. 2011, 6, pages 138-149.

Methods of analysis of the platelet aggregation in whole blood are also known using measuring of the electrical resistance between two electrodes immersed in the whole blood, described for example in D. C. Cardinal, R. J. Flower, “The electronic aggregometer: A novel device for assessing platelet behavior in blood”, J. Pharmacol. Methods, 1980, 3, 135-158.

Analyses methods in vivo of phenomena of stagnation of the blood are also known (see: T. Dai, A. Adler, “In vivo blood characterization from bioimpedance spectroscopy of blood pooling”, IEEE Trans. Instr. Meas., 2009, 58, 3831-3838) and to monitor the viscosity of blood during heart surgery operations using impedance spectroscopy techniques (see G. A. M. Pop, T. L. M. de Backer, M. de Jong, P. C. Struijk, L. Moraru, Z. Chang, H. G. Goovaerts, C. J. Slager, A. J. J. C. Bogers, “On-line electrical impedance measurement for monitoring blood viscosity during on pump heart surgery”, Eur. Surgical Res., 2004, 36, 259-265).

The Cole-Cole parameters of induced spectral polarization detected using impedance spectroscopy can be put in correlation with the hematocrit (see: Y. Ulgen, M. Sezdi, “Physiological quality assessment of stored whole blood by means of electric measurements”, Med. Bio. Eng. Comput., 2007, 45, 653-660), with the quality of the blood (see: Y. Ulgen, M. Sezdi, “Hematocrit dependence of the Cole-Cole parameters of human blood”, IEEE Proc. 2nd Int. Biomed. Eng. Days, 1998, 71-74), and with the viscosity.

In particular, a logarithmic increase (exponential) can be seen of the capacity associated to the cellular membrane as the fibrinogen increases (respectively of the hematocrit). Because of the insulating membrane of the red blood cells, which is non-conductive under 20 kHz, the electric resistance of the whole blood at low frequencies greatly depends on the hematocrit as described in T. Yamagata, H. Fujisaki, “Investigation of electrical resistivity changes of human blood during dynamic exercise”, Trans. Electrical and Electronic Engineering, IEEJ 2008, 3, 79-83. Above 10 MHz the membrane behaves as a short circuit and the conductance measured is a weighed average of the conductivity associated to the plasma and to the intracellular fluid.

A quantitative model of the conductivity of the blood in conditions of laminar flow stationary inside rigid cylindrical tubes is described in A. E. Hoetink, T. J. C. Faes, K. R. Visser, R. M. Heethaar, “On the flow dependency of the electrical conductivity of blood”, IEEE Trans. Biomed. Eng. 2004, 51, 1251-1261, or a model based on an extension of the Maxwell-Fricke theory which takes into account the orientation and deformation effect of the ellipsoidal particles which are induced by the shearing force; an extension of this analysis to include also a pulsatile flow is described in R. L. Gaw, B. H. Cornish, B. J. Thomas, “The Electrical Impedance of Pulsatile Blood Flowing Through Rigid Tubes: A Theoretical Investigation”, IEEE Trans. Biomed. Eng. 2008, 55, 721-727.

In K. Asami, K. Sekine, “Dielectric modelling of erythrocyte aggregation in blood”, J. Phys. D: Appl. Phys. 2007, 40, 21972204, the dielectric behavior of whole blood is simulated using an erythrocyte model that consists of a disc covered by a membrane, and the model of an aggregate of erythrocytes consists of a pile of discs with regular spacing.

Moreover, in O. Baskurt, M. Uyuklu, S. Ozdem and H. J. Meiselman, “Measurement of red blood cell aggregation in disposable capillary tubes”, Clinical Hemorheology and Microcirculation 47, pages 295-305, 2011, and in O. Baskurt, M. Uyuklu and H. J. Meiselman, “Simultaneous monitoring of electrical conductance and light transmittance during red blood cell aggregation”, Biorheology 46, pages 239-249, 2009, techniques were experimented which measure and correlate electrical conductance (by means of two electrodes positioned at beginning and end of the channel) and light transmittance transverse to the fluid channel where the erythrocytes are aggregated per shear rate of 5001/s per 75 sec.

In O. Kwon, J. K. Seo, E. J. Woo and J-R. Yoon, “Electrical Impedance Imaging for Searching anomalies”, Comm. Korean Math. Soc. 16 (2001), No. 3, pages 459-485, the reconstruction is evaluated, from the point of view of inverse problems, in a two-dimensional disc of the geometry (position and sizes) of a limited number of 2D defects, with a different conductivity from that of the disc, starting from the measurement of potentials in a high number of points (>30) distributed uniformly along the whole edge of the domain. At the same points a distribution of current is also applied, prescribed by near Neumann conditions (that is, on the normal derivative at the edge of the potential) so as to guarantee a sum of nil and uniform density in the disc. The potentials are also normalized so as to guarantee nil average.

In recent times, considerable efforts have been made and increasing attention has been paid to designing microfluidic devices with different geometries, for example which can be personalized at the user's request, both of channels with minimum sizes of a few microns, and also electrodes with minimum sizes of 10 microns, such as blood cell counters (on this point see N. Piacentini, D. Demarchi, P. Civera, M. Knaflitz, “Blood Cell Counting by Means of Impedance Measurements in a Microsystem Device”, Proc. 30th Int. IEEE EMBS Conf. 2008, 4824-4827) or cell separators (on this point see Y. Nakashima, S. Hata, T. Yasuda, “Blood plasma separation and extraction from a minute amount of blood using dielectrophoretic and capillary forces”, Sens. Actuators B 2010, 145, 561-569), so that various suppliers have developed the microtechnologies that allow these to be produced.

These technologies for making the channels and the electrodes integrated therein easily allow vision under the conventional optical or fluorescence microscope, inverted or confocal, and to measure the electrical behavior of an impedenziometric type using commercial instruments for measuring impedance (LCR Meter Agilent HP4284A class <0.5%) or developed ad hoc.

An automatic method is also known for monitoring cell behavior, allowing to observe, for example, cell proliferation, cell adhesion and the spread of the adhesion, mobility, and the poration of the cell membranes. The method consists of a set of electrodes printed on a gold film, powered by alternate tensions and currents at variable frequency and amplitude; between pairs of electrodes (circular in shape and with a diameter of 250 micron) the module Z of impedance is measured and, in other methods, also the module and phase of the impedance. Given that the cell membranes at the frequencies of use (<200 kHz) are practically insulating, morphological changes of the cell disposition modify the pathways of the electrical currents, causing a consequent variation in the impedance measured at the pairs of electrodes.

There are different electrode matrices, each with a diameter of 250 micron, both for stationary cell cultures and in perfusion conditions with a high shear rate with a channel 50 mm in length, 5 mm in width and 0.4 mm in height.

A method and apparatus is also known from the publication by A. Affanni, R. Specogna and F. Trevisan, “Measurement Bench for impedence Tomography during Hemostasis Process in whole Blood”, which are able to detect only the impedance of platelet aggregates. In particular, a device is made, also called perfusion chamber, provided with a base plate on which at least one micro-channel is made and a cover plate or slide that is put above the base plate to close the micro-channel.

On the cover plate, and in particular on the surface facing toward the micro-channel during use, a plurality of electrodes are made, disposed parallel to each other. The electrodes have a mainly oblong configuration, that is, they have a length much greater than their width. The electrodes are disposed during use in a direction substantially orthogonal to the development of the micro-channel.

The electrodes are in turn connected to a current generator and during the measuring steps the tension on the electrodes is measured. Depending on the measurements made, it is possible to determine the impedance of the blood.

With this device, however, it is not possible to determine the volume of aggregates in a biological fluid. In order to evaluate the volume, it is necessary to correlate at least the impedance data detected with this method with optical information detected with optical acquisition devices. This makes the method and apparatus particularly complex and difficult to use in the surgery or for large-scale analyses.

Complete kits are also known, with a total of 10 fluid and electrical outlets, able to be personalized and studied for both fluorescence and traditional microscopy, with closed channels of variable length, variable heights from some tens of micron and widths in the range of 100 micron.

Solutions are also known in which optical fibers are associated with the microfluidic channel through which the blood passes, in order to illuminate and receive the light emitted in a focused manner.

Regarding the detection of electrical quantities for analyzing processes affecting the blood, the documents KR2008015212, SU1278697 and US-B2-6797150 are known. The first uses two electrodes in contact with the stationary blood in a cylindrical container and the impedance is measured and acquired. The second identifies the formation of spatial coagulation structures of the blood by comparing measurements of conductivity continuously and in frequency, and the prevalence of continuous conductivity over frequency conductivity indicates the event. The third, measuring capacity and resistance in the domain of time and in frequency, evaluates the concentration of glucose, the hematocrit etc. of a biological sample, for example blood, which occupies the whole volume between two electrodes of an electrochemical cell characterized by a pair of flat electrodes, facing each other and distanced by 1″. The document indicates with “S” the conductor area in contact with the electrodes of the cell sample, capacity “C” depends on “S” (if the series capacity of the double layer of ions at the interface between electrodes and cells is constant), resistance R depends on 1/S, their C/R ratio is proportional to the square of “S”. From this ratio the area covered by the sample is deduced, and hence the volume Sx1.

Document US-A1-20080297169 provides to measure a fraction of particles in biological fluids (for example hematocrit in the blood, coagulation speed, prothrombin time PT, activated partial thromboplastin time APPT, activated clotting time ACT, thrombin clotting time TCT) and non-biological fluids. It also allows to determine the concentration of an analyte in a blood sample, plasma, serum, urine etc. The fluids are stationary. The device contains the necessary reagents. The technique is based on measuring impedance in AC or resistance in DC between pairs of electrodes. A standard thermostatic system keeps the temperature for example at 35° C. Electrical feeds are standard, as is the system for acquiring electrical signals of tension and current in order to calculate resistance or impedance.

Document WO-A1-2005114140 describes a device for measuring the clotting of whole blood or the prothrombin time in serum. It studies the motion of a particle made of ferromagnetic material driven by magnetic forces inside the chamber containing the sample of biological fluid to be examined.

Finally, document WO-A1-2011073481 describes a multi-electrode system which measures the change in impedance due to the presence of a particular component in a solution of a different nature (biological or otherwise). The change in impedance is connected to the particular component present.

The documents cited above relating to impedenziometric measurements are not able to determine with reasonable certainty the evolution of the growth of a platelet aggregate in flow conditions of the hemostatic fluid, but only when the hemostatic fluid is stationary, for example in a test tube.

This analysis of the hemostasis process might be achievable with optical techniques but the equipment required is extremely complex to manage and use, and is also particularly expensive, to such an extent that using it in a surgery is not justified.

One purpose of the present invention is to obtain an apparatus for analyzing the process of aggregate formation in a biological fluid, such as blood, in flow conditions, in order to estimate the development over time of the growth of the aggregate, that is, its volume.

Another purpose of the present invention is to obtain an apparatus that is reliable and precise and that allows to acquire, substantially in real time, and to analyze the aggregate process in its entirety and complexity with a high degree of reliability and precision.

Another purpose of the present invention is to obtain an apparatus for analyzing the aggregation process in a biological fluid that is economical, easily transported and also suitable to be used in surgery type structures.

Another purpose of the present invention is to perfect a method for analyzing the process of aggregate formation in a biological fluid, such as blood, in flow conditions, which is simple, quick, reliable and economical.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purposes, an apparatus according to the present invention for analyzing the process of formation of aggregates in a biological fluid, such as blood or hematic fluids, comprises a perfusion chamber provided with at least one micro-channel through which the biological fluid flows, and in which at least one reactive substrate is present, such as a cyto-adhesive substrate, to stimulate the aggregation process in the biological fluid. Impedenziometric detection means are associated with the micro-channel, disposed in correspondence to at least one investigation area and, during use, in contact with the flow of biological fluid in transit, in order to detect impedance data of the biological fluid.

According to one feature of the present invention, the impedenziometric detection means comprise at least two first electrodes disposed at least partly in the micro-channel, distanced from and facing each other, and a plurality of second electrodes disposed reciprocally so as to define the perimeter of a closed surface zone contained in the micro-channel.

The second electrodes are disposed between the first electrodes.

Forms of embodiment of the present invention provide that the surface area has a shape chosen from a polygon such as a hexagon, a decagon or, in other forms of embodiment, a circumference. In this latter case the second electrodes have an arched conformation.

According to a possible form of embodiment the first electrodes and/or the second electrodes have an oblong development, that is, they have a length that is much greater than their width.

The detection of the impedenziometric type can also be associated to the use of optical acquisition means which, if present, can also be used for a possible validation of the data acquired by the impedenziometric detection means. In this way, it is possible to obtain a simple apparatus, inexpensive, and which can also be used in outpatient care and not only for research.

In particular, the invention provides to electrically feed the first electrodes and to detect electrical quantities induced by the first electrodes in the second electrodes so as to determine, by processing said electrical quantities, the volume of aggregate which slowly forms in the investigation area associated to the micro-channel.

According to some forms of embodiment of the present invention, the first electrodes are disposed substantially parallel to each other. This configuration allows to achieve a uniform field during the execution of the measurements.

According to other forms of embodiment, the first electrodes are disposed transverse to the longitudinal development of the micro-channel.

According to one possible form of embodiment, the first electrodes are connected to a current generator and provide to feed the first electrodes, or current electrodes, with a sinusoidal electric current in order to subsequently detect a tension, or a potential, at the heads of each of the second electrodes, or potential electrodes. By suitably adding together the individual electrical potentials detected in the second electrodes it is possible to identify a direct correlation with the volume of the aggregate that is formed in the investigation area.

According to another feature of the invention, the apparatus also comprises electrical feed devices and devices to measure the electric quantities connected to the first electrodes and/or to the second electrodes.

According to another form of embodiment, at least the electrical feed devices and the measuring devices are associated to a processing unit to control, command and acquire data and process data.

### BRIEF DESCRIPTION OF THE DRAWINGS

These and other characteristics of the present invention will become apparent from the following description of one form of embodiment, given as a non-restrictive example with reference to the attached drawings wherein:

FIG. 1 is a schematic representation of an apparatus for analyzing the process of formation of aggregates according to the present invention;

FIG. 2 is a plan view of a detail in FIG. 1;

FIG. 3 is a perspective view of a component of the detail in FIG. 2;

FIG. 4 is a view of a detail in FIG. 2;

FIG. 5 is a variant of FIG. 4;

FIG. 6a is an enlarged detail in FIG. 5;

FIG. 6b is a variant of FIG. 6a;

FIG. 7 is a graph that shows the development of the impedance measured as a function of the volume of the platelet aggregate;

FIG. 8 is a schematic representation of a variant of FIG. 5;

FIG. 9 is a graphic representation by means of isovalue lines of the uniformity of the sensitivity of the system as the position of the defect varies inside the acquisition zone.

To facilitate comprehension, the same reference numbers have been used, where possible, to identify identical common elements in the drawings. It is understood that elements and characteristics of one form of embodiment can conveniently be incorporated into other forms of embodiment without further clarifications.

### DETAILED DESCRIPTION OF SOME FORMS OF EMBODIMENT

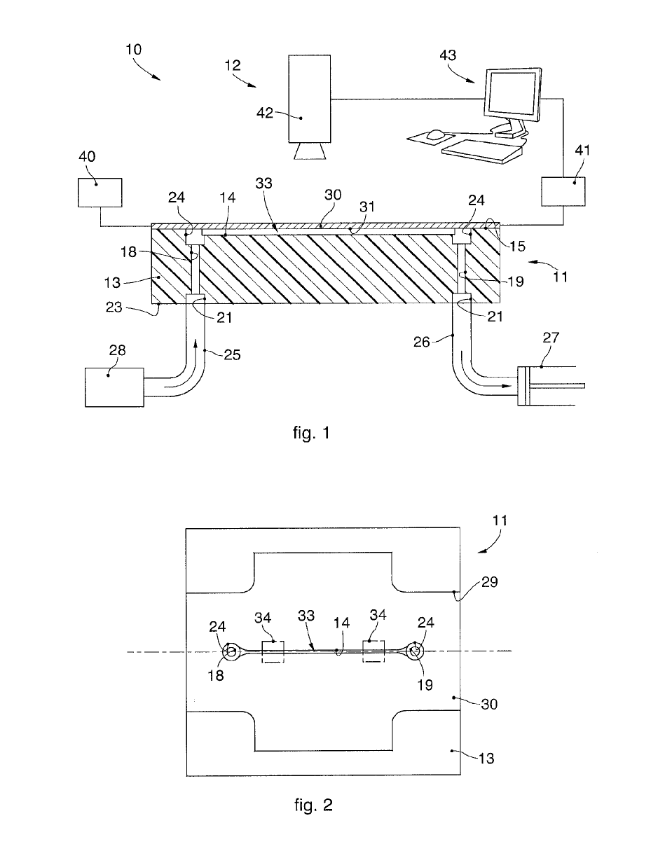

With reference to FIG. 1, an apparatus for analyzing the process of aggregate formation in a biological fluid according to the present invention is indicated in its entirety by the reference number 10, and comprises at least a perfusion chamber 11 in which a biological fluid, in this case blood, is made to flow.

Hereafter in the description we shall refer to the specific case of the formation of platelet aggregates, but it is quite evident that the description is equally valid for other aggregates such as aggregates of red corpuscles or suchlike.

The fluid to be analyzed can be biological fluid, blood, hematic fluids both animal or human, and mixtures of said fluid with additive substances.

The perfusion chamber 11 (FIGS. 1, 2 and 3) comprises a base body 13 provided with at least one micro-groove 14 made on a first surface 15 of the base body 13, and having a mainly longitudinal development and a rectangular section, merely by way of example 0.4 mm in width and 0.2 mm in height.

In correspondence with each of the terminal ends of the micro-groove 14, and orthogonal to the thickness of the base body 13, an inlet channel 18 is made, and respectively an outlet channel 19, for the blood that is made to flow through the micro-groove 14.

The inlet channel 18 and outlet channel 19 have a connection end 21 (FIG. 1) made in correspondence with a second surface 23 of the base body 13, which is opposite the first surface 15, and a flared end 24 that opens toward the micro-groove 14.

In correspondence with the connection end 21 of the inlet channel 18, an inlet pipe 25 for the blood is connected, whereas in correspondence with the outlet channel 19 an outlet pipe 26 for the blood is connected.

An element 28 for containing the blood is associated with the inlet pipe 25, whereas a pumping device is associated with the outlet pipe 26, in this case a syringe pump 27, which provides to aspirate the blood from the inlet pipe 25, making it pass through the micro-groove 14 with a constant flow rate and a speed gradient, or shear rate, not less than 3000 ŝ-1.

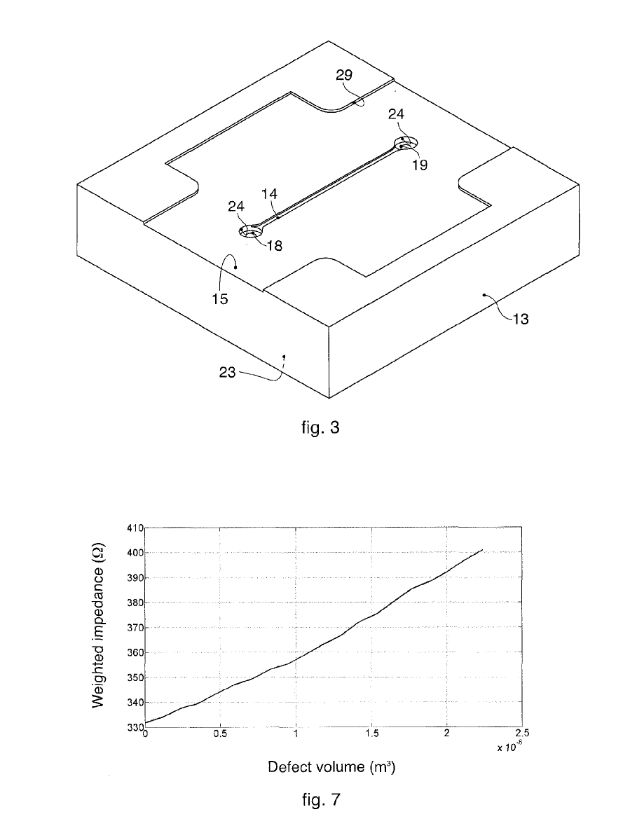

The first surface 15 (FIGS. 2 and 3) is defined by a shaped housing seating 29, made recessed in the overall thickness of the base body 13.

The housing seating 29 is configured to allow the stable positioning and precise housing of a cover element, in this case a slide 30 (FIG. 1), which is put in contact with a support surface 31 against the first surface 15 of the base body 13 and provides to close the micro-groove 14 in order to define a micro-channel 33 for the passage of the blood.

The first surface 15 of the base body 13 and the support surface 31 of the slide 30 have very restricted geometric and dimensional tolerances, to guarantee the correct reciprocal planarity between them and guarantee the air-tight seal of the micro-channel 33.

The syringe pump 27 generates a depression in the micro-channel 33 which not only aspirates the blood from the containing element 28 but also provides to maintain the base body 13 and the slide 30 adherent to each other.

On the support surface 31 of the slide 30, before the analysis, a cyto-adhesive substance is uniformly distributed, such as a collagen, to promote the platelet aggregation of the blood.

Attachment means of a known type, not shown in the drawings, are provided to reciprocally attach the base body 13 and the slide 30.

The perfusion chamber 11 is typically made of transparent material and its geometry must be suitable to simulate the desired fluid-dynamic conditions, such as for example the shear rate and the laminar flow.

In particular, the perfusion chamber 11 must guarantee a good hydraulic seal, in order to preserve the desired fluid-dynamic conditions and not negatively affect the reliability of the analysis.

The material used to make the perfusion chamber 11, that is, for the base body 13 and the slide 30, is chosen from a group comprising glass, COC (cyclic olefin copolymers), COP (cyclic olefin polymers), high optical grade polycarbonate, casting silicone, or polydimethylsiloxane, also known as PDMS or suchlike.

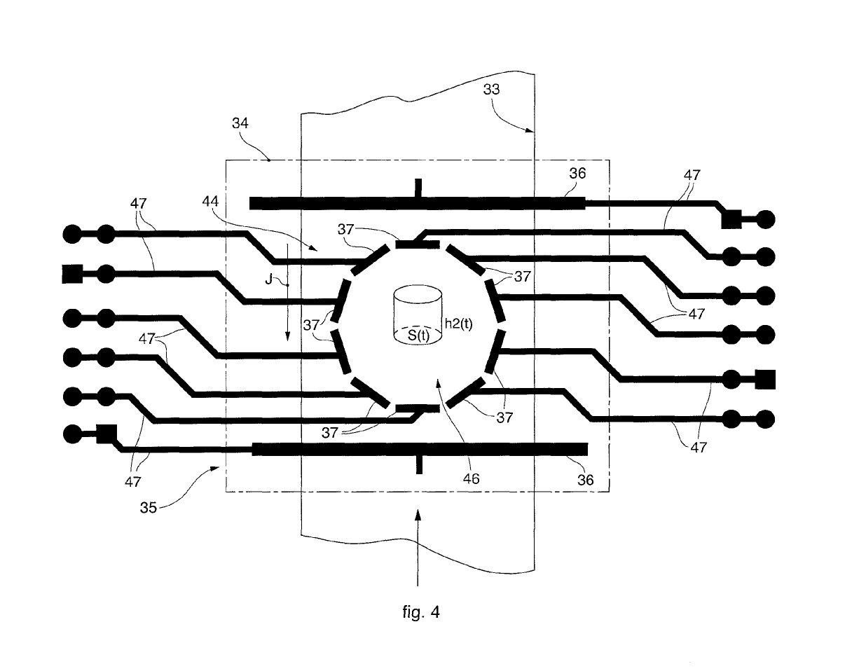

On the support surface 31 of the slide 30 and in investigation areas 34, in this case two investigation areas 34, of the same support surface 31 which is disposed in correspondence with the micro-channel 33, there are impedenziometric detection means 35 (FIG. 4), which provide to perform an impedenziometric measurement of the platelet aggregate which is gradually formed inside the micro-channel 33.

Merely by way of example, each investigation area 34 is about 0.2 mm wide and 0.2 mm long.

A first form of embodiment of the present invention (FIG. 4) provides that the impedenziometric detection means 35 comprise two first electrodes 36 disposed at least partly in the micro-channel 33, distanced from and facing each other.

According to possible forms of embodiment, the first electrodes 36 can be disposed parallel to each other and transverse to the longitudinal development of the micro-channel 33.

In possible forms of embodiment, the first electrodes 36 can have an oblong development, that is, with a width that is much less than the length.

Merely by way of example, the first electrodes 36 are about 250 μm long and are reciprocally distanced by about 200 μm.

The first electrodes 36 delimit between them, possibly also with the lateral walls of the micro-channel 33, a first zone 44 of the micro-channel 33 through which the blood passes.

The impedenziometric detection means 35 also comprise a plurality of second electrodes 37 defining with each other the perimeter of a closed surface zone, or second zone 46, contained inside the micro-channel 33.

In particular, the second electrodes 37 are disposed between the first electrodes 36, or, in possible forms of embodiment, inside the first zone 44.

The second zone 46 is contained in the first zone 44 and defines the inspection area for analyzing the process of aggregate formation.

In possible forms of embodiment, the second electrodes 37 can have a mainly oblong development.

However, it is not excluded that in other forms of embodiment the second electrodes 37 may have a different shape, provided that they have at least one perimeter portion, or perimeter edge, which is disposed on the perimeter of the second zone 46.

According to one form of embodiment, the second electrodes 37 have a substantially rectilinear development and are disposed along the sides of a polygon.

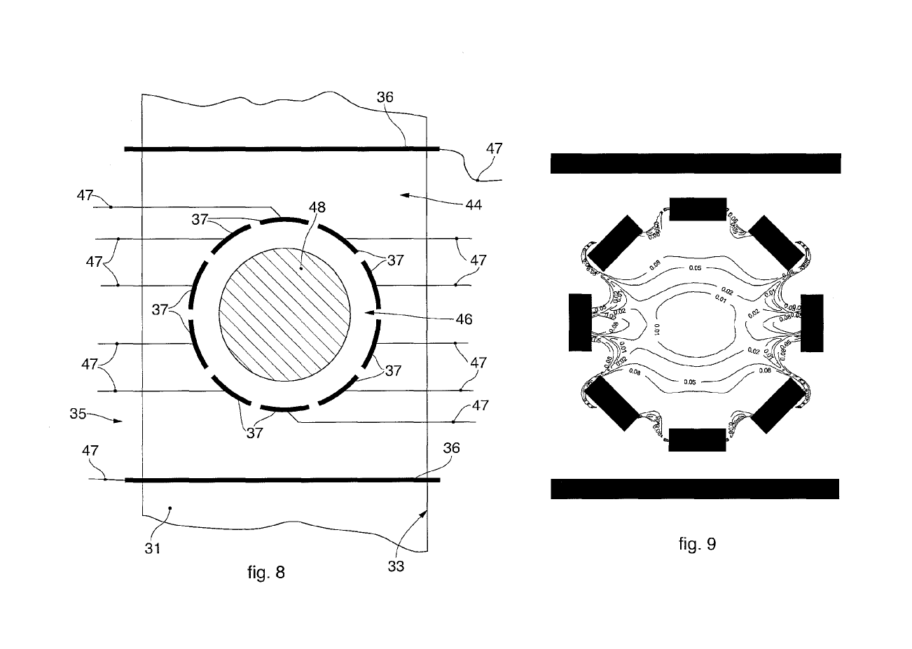

According to another form of embodiment, shown in FIG. 8, the second electrodes 37 each have an arched conformation and are disposed so as to define a circumference.

In the form of embodiment shown in FIG. 4, the impedenziometric detection means 35 comprise ten second electrodes 37, with a rectilinear development and disposed along the sides of a decagon, although it is not excluded that, in other forms of embodiment, the second electrodes 37 may be more or fewer in number than ten.

Other forms of embodiment provide that each of the second electrodes 37 is angled with respect to the adjacent second electrode 37 by a constant angle.

Another form of embodiment provides that there is an even number of second electrodes 37, preferably a non-multiple of four. In fact, a non-multiple of four of second electrodes 37 ensures that each useful contribution of current—Neumann's condition—is not nil; the useful current for the nth electrode is given by the equation [symbol] , where J is the density of electrical charge, n is the versor perpendicular to the electrode in the plane of the slide and S is the area obtained by multiplying the width of the electrodes by a unitary depth.

Another form of embodiment provides that two of the second electrodes 37 are disposed substantially parallel to each other and parallel respectively to the first electrodes 36.

According to another form of embodiment, the second electrodes 37 are all substantially the same length, in order to ensure an equal area exposed to the electrical current for each of the second electrodes 37.

The first electrodes 36 and the second electrodes 37 are made of conductive material, chosen from at least gold, platinum and indium-tin oxide, or indium oxide doped with tin, also known by the acronym ITO.

The first electrodes 36 and the second electrodes 37 are made by molding on the support surface 31, for example using deposition techniques with material evaporation (PVD), sputtering techniques or other similar or comparable techniques.

The first electrodes 36 and the second electrodes 37 comprise an active sensitive part which, during use, is in direct contact with the blood, and the remaining part of the electrode that is insulated, for example using passivization techniques.

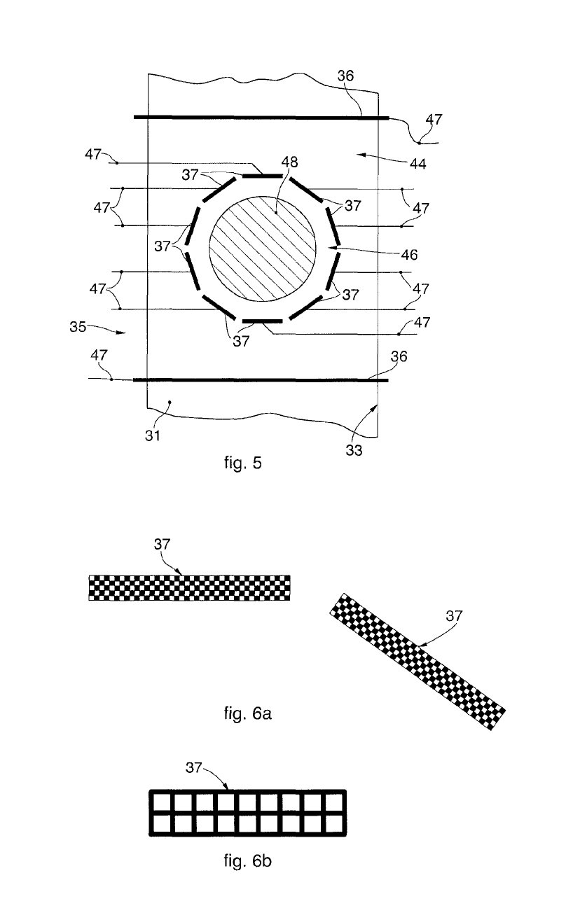

Both the first electrodes 36 and the second electrodes 37 are connected by conductive tracks 47 (FIGS. 4 and 5), independently of each other, to electrical feed devices 40 (FIG. 1) of a known type, and to devices 41 to measure the electrical quantities.

According to a possible form of embodiment, the first electrodes 36 are connected to devices to feed an electrical current.

According to another form of embodiment, the second electrodes 37 are connected to a device to measure tension.

Other forms of embodiment provide that the first electrodes 36 and the second electrodes 37, or at least some of them, are each made according to a chess-board (FIG. 6a) or matrix (FIG. 6b) configuration. In particular, in FIG. 6a it is provided that the region affected by the second electrode 37 is divided into a plurality of lines and columns to define a plurality of boxes. The chess-board thus defined will have an alternation of first regions or boxes affected by conductive material that make up the electrode, and an alternation of second regions or boxes, each disposed adjacent to every side of the first boxes, and coated with a water-repellent coating. In FIG. 6b the second electrode 37 comprises first regions affected by the conductive material that makes up the electrode and second regions comprising lines disposed transverse and parallel to each other in a matrix configuration and which delimit the first zones. This particular configuration allows to prevent the problem of the formation of aggregates on the electrodes 36, 37, since the aggregate will tend to preferably adhere to the water-repellent part. The advantage of this configuration is that it is possible to reduce the adhesion to the electrode, while the limit of the sizes of each cell is imposed by the technology available. The water-repellent material, merely by way of example, can be a polytetrafluoroethylene, also known by its acronym PTFE.

The support surface 31 (FIG. 5) of the slide 30 is suitably treated with a water-repellent coating 48 disposed substantially in a zone comprised inside the second electrodes 37, that is, inside the second zone 46. The water-repellent coating 48 allows to obtain a preferential zone in which the platelet thrombus attaches itself.

According to some forms of embodiment, it may be provided that the water-repellent coating 48 is made of a polymer material chosen from a group comprising polytetrafluoroethylene, also known by its acronym PTFE.

Merely by way of example, the water-repellent coating 48 has a thickness comprised between 10 nm and 30 nm.

The apparatus 10 according to the present invention may provide a possible optical acquisition device 12 which allows to identify the position and formation geometry of the aggregates inside the investigation area 34 of the micro-channel 33. The optical acquisition device 12 can be used to validate the results that are detected by the impedenziometric detection means 35.

In this case the optical acquisition device 12 comprises an optical module 42 for epifluorescence analysis in order to highlight the platelets that are marked with a suitable fluorescent probe to adhere to the cyto-adhesive substrate.

The optical module 42 can be the fluorescence type with or without lenses of the two-dimensional type.

The electrical feed devices 40, the measuring devices 41 and possibly the optical acquisition devices 12 are associated to a processing unit 43 provided to control, command, acquire data, and process data supplied by the latter.

The apparatus 10 according to the present invention can advantageously comprise thermostating means to keep the perfusion chamber 11 at a desired temperature, advantageously at the physiological temperature which in the case of the human body is about 37° C.

We shall now describe a method to analyze the process of platelet aggregation using an apparatus 10 as described above.

The method comprises at least a step in which a hematic fluid sample is taken from a patient and suitably prepared in ways that vary depending on the tests to be carried out.

More specifically, at least one sample of blood from the veins is required, the blood must be temporarily preserved in a test tube containing anti-clotting substances such as for example heparin, citrate or suchlike, and fluorescent substances are possibly added, for example quinacrine and antibody anti-fibrin phycoerythrin.

Then, a cyto-adhesive substance is deposited on the support surface 31 of the slide 30, and the slide 30 is subsequently positioned on the base body 13.

Afterward, the blood is circulated through the micro-channel 33 by driving the syringe pump 27, in order to trigger the phenomenon of platelet aggregation which, merely by way of example, can last about 6 minutes.

At this point, the analysis of the platelet aggregation process is started, which provides an impedenziometric measuring step, using the measuring devices 41, and a processing substep using the processing unit 43, during which the information acquired by the impedenziometric detection means 35 is correlated.

The impedenziometric measuring step provides that between the first electrodes 36, also called current electrodes, a sinusoidal current is applied with a frequency f<200 kHz that generates a uniform current density J by translation inside the polygon defined by the second electrodes 37, or potential electrodes. The first electrodes 36 are configured so that the current density inside the second zone 46 is uniform.

It is then provided to measure the potentials of each of the second electrodes 37 forming the polygon, deducing the electrical tensions with respect to a reference electrode; the useful current for the nth electrode is given by the formula [symbol] with J current density, n versor perpendicular to the electrode in the plane of the slide and S the area obtained by multiplying the width of the electrode by a unitary depth. The measurement signal is obtained by adding together the products of the tension and current useful for the nth electrode; the signal measured is constant, given the same volume of the aggregate inside the second zone 46. This implies that, irrespective of the position where the aggregate forms, the measurement signal is proportionate to the volume occupied thereby.

FIG. 7 shows the linearity of the system, progressively inserting volume defects 1.18 10⁻⁹ m³ inside the polygon defined by the second electrodes 37, the electrical properties of which are analogous to the platelet thrombus at the frequencies that concern us.

As can be seen, the signal measured is linear as the volume of the defect increases; this means that, irrespective of where the defect forms, the signal measured does not vary.

Given the linear relationship between the signal measured and the volume of the defect, in theory the present invention could lead to a measuring apparatus that does not necessarily need optical information.

More specifically, while the potentials are being measured at the heads of the second electrodes 37 (FIG. 4), the nth second electrode 37 is affected by the useful current I, which depends on the current density J and on the orientation of the electrode itself in plane x, y defined by the support surface 31 of the slide 30 according to the formula [symbol] where n is the versor perpendicular to the electrode in the plane of the slide.

The group of second electrodes 37 can be approximated as an n-pole where, with a good approximation, the following formula applies:

[symbol]

One of the second electrodes 37 is assumed as the reference electrode and the tension Uᵢ of the nth electrode is measured at instant “t”, with i=1, . . . , N. From the measurements of tension and useful current the power [symbol] , relating to the same instant entering into the n-pole, is calculated.

Applicants have investigated the uniform sensitivity of this system, not only experimentally but by means of numerical simulation, as the volume of the defect and its position vary. To this purpose a prototype was made, on a macroscopic scale (40:1) to verify this property, and the configuration of the system was simulated in the presence of a cylindrical defect with a diameter of 1 mm and a height of 2 mm which moves on a grid of 11×14 points spaced at 1 mm.

FIG. 9 is a graph using isovalue lines of the uniform sensitivity of the system as the position of the defect varies. In particular, the quantity represented by the isovalue lines in one point is the relative variation expressed in a percentage between the power entering into the n-pole when a certain defect is positioned in said point with respect to the power which is obtained when the same defect is in the center of the second zone 46.

From these simulations it is clear that inside the second zone 46 the sensitivity is practically uniform, whereas in the external region there are zones that over-estimate or under-estimate the volume. To obviate this problem, it is useful to position the cyto-adhesive substance only in the region inside the second zone 46 defined by the second electrodes 37, for example, using a mask and/or micro-pipettes, and to make the electrodes 36, 37 according to a matrix or chess-board configuration as described above.

It is clear that modifications and/or additions of parts may be made to the apparatus for the analysis of the aggregate formation process in a biological fluid and the corresponding method of analysis as described heretofore, without departing from the field and scope of the present invention.

It is also clear that, although the present invention has been described with reference to some specific examples, a person of skill in the art shall certainly be able to achieve many other equivalent forms of apparatus for the analysis of the aggregate formation process in a biological fluid and the corresponding method of analysis, having the characteristics as set forth in the claims and hence all coming within the field of protection defined thereby.

1. Apparatus for analyzing the process of formation of aggregates in a biological fluid, such as blood or hematic fluids, comprising a perfusion chamber provided with at least one micro-channel through which said biological fluid flows, and in which at least one reactive substrate is present, such as a cyto-adhesive substrate, to stimulate the aggregation process in the biological fluid, and impedenziometric detection means associated with said micro-channel, in correspondence to at least one investigation area and disposed, during use, in contact with the flow of biological fluid in transit, in order to detect impedance data of said biological fluid, wherein said impedenziometric detection means comprise at least two first electrodes disposed at least partly in said micro-channel distanced from and facing each other, and a plurality of second electrodes reciprocally disposed so as to define the perimeter of a closed surface zone contained in said micro-channel, said second electrodes being disposed between said first electrodes.

2. Apparatus as in claim 1, wherein said first electrodes are disposed parallel to each other.

3. Apparatus as in claim 1, wherein said first electrodes are disposed transversely to the longitudinal development of said micro-channel.

4. Apparatus as in claim 1, wherein said second electrodes have a substantially rectilinear development and are disposed according to a pattern at the sides of a polygon.

5. Apparatus as in claim 1, wherein said second electrodes each have an arched conformation and are disposed so as to define the perimeter of a circumference.

6. Apparatus as in claim 1, wherein it comprises electrical feed devices and measuring devices to measure the electric quantities connected to said first electrodes and/or said second electrodes.

7. Apparatus as in claim 1, wherein said first electrodes are connected to electric current feed devices and in that said second electrodes are connected to a tension measuring device.

8. Apparatus as in claim 1, wherein the number of said second electrodes is an even number.

9. Apparatus as in claim 1, wherein two of said second electrodes are disposed substantially parallel with respect to each other and parallel respectively to said first electrodes.

10. Apparatus as in claim 1, wherein said second electrodes all have the same length.

11. Apparatus as claim 1, wherein said micro-channel has at least one surface on which said impedenziometric detection means are disposed, and in that said support surface, in the zone comprised inside said surface zone, is treated with a water-repellent coating.

12. Apparatus as in claim 1, wherein at least one of either said first electrodes or said second electrodes is made according to a matrix configuration provided with an alternation of first boxes affected by conductive material and an alternation of second boxes each disposed adjacent to each side of the first boxes and each affected by a water-repellent coating.

13. Method for analyzing the process of formation of aggregates in a biological fluid, such as blood or hematic fluids, comprising at least a step in which said biological fluid is made to flow through at least one micro-channel of a perfusion chamber, a reactive substrate also being present in said micro-channel, such as a cyto-adhesive substrate, to stimulate an aggregation process in said biological fluid, and an impedenziometric measuring step, by means of the measuring devices associated to said micro-channel in correspondence to at least one investigation area, during which the information acquired by the impedenziometric detection means is correlated, wherein it provides:
to electrically feed at least two first electrodes disposed at least partly in said micro-channel distanced from and facing each other,
to detect electrical quantities in a plurality of second electrodes reciprocally disposed so as to define the perimeter of a closed surface zone contained in said micro-channel, said second electrodes being disposed between said first electrodes, and
to process said electrical quantities to determine the volume of the aggregate in said investigation area.

14. Method as in claim 13, wherein said first electrodes are fed with a sinusoidal electric current to generate a uniform density of current inside the geometric figure defined by the second electrodes.

15. Method as in claim 14, wherein said sinusoidal electric current has a frequency less than 200 kHz.

16. Method as in claim 13, wherein in each of said second electrodes the potential is measured.
